(19) United States
(12) Patent Application Publication
(10) Pub. No.: US 2009/0074703 A1
(43) Pub. Date: Mar. 19, 2009

(54) ANIMAL BEDDING MATERIAL

(76) Inventor: Charlene Jewell, Martinsville, IN (US)

Correspondence Address: BAKER & DANIELS LLP, 300 NORTH MERIDIAN STREET, SUITE 2700, INDIANAPOLIS, IN, 46204, US

(21) Appl. No.: 12/209,313

(22) Filed: Sep. 12, 2008

Related U.S. Application Data
(60) Provisional application No. 60/972,266, filed on Sep. 14, 2007.

(51) Int. Cl. A61L 9/01; B02C 19/00

(52) U.S. Cl. 424/76.1; 424/76.8; 241/101.6

(57) ABSTRACT

The present disclosure includes an animal bedding material comprising an aggregate of sized paper and corrugated material. The present disclosure also includes a method of generating animal bedding material comprising the steps of sizing paper, sizing corrugated material and aggregating the paper and the corrugated material.

### CROSS REFERENCE TO RELATED APPLICATIONS

This application claims the benefit of U.S. Provisional Application No. 60/972,266, filed Sep. 14, 2007, the disclosure of which is expressly incorporated by reference.

### FIELD

This application relates to animal bedding material.

### BACKGROUND

Animal bedding material, such as horse bedding material, has typically comprised of a number of different materials. Traditionally, horses and other animals have bedded on straw, various wood products such as wood shavings and sawdust. Straw, wood shavings and sawdust have also been described in U.S. Pat. No. 5,152,251 to Aukeman et. al. This application expressly incorporates by reference Aukeman. Straw, sawdust, and wood shavings are known to have a high percentage of fine, particulate matter. This tends to increase dust generation which can be possibly harmful to the animal, such as to the respiratory system of the animal.

### SUMMARY

The present disclosure relates to an improved product and process for making an animal bedding material particularly suited for use with horses or particularly this disclosure relates to animal bedding material comprising an aggregate of corrugated material and paper, wherein the paper and corrugated material is sized to minimize fine particle generation and to provide for efficient absorption of animal waste.

The present disclosure also includes a method of generating animal bedding material comprising the steps of sizing paper, sizing corrugated material and aggregating the paper and the corrugated material.

The present disclose also relates to an apparatus for providing an animal bedding material, comprising a first conveyor configured to transfer a corrugated material, a first shredder adjacent to the first conveyor, the first shedder configured to strip cut and cross cut the corrugated material, a pneumatic pick up hood adjacent to the first shredder, the pneumatic pick up hood configured to convey the corrugated material, at least one classifier located in relation to the pneumatic pick up hood, the classifier configured to separate the corrugated material based on the size of the corrugated material, wherein the classifier utilizes a walking classifier screening process configured to separate sized corrugated material from large corrugated material, the walking classifier screening process configured to twice separate the sized corrugated material from the large corrugated material, wherein the classifier includes a filter configured to separate the sized corrugated material from fines and dust, an large corrugated material rotary pressure valve located in relation to the at least one classifier, the large corrugated material rotary pressure valve configured to collect the large corrugated material, an large corrugated material storage bin for storing the large corrugated material, a sized corrugated material rotary pressure valve located in relation to the at least one classifier, the sized corrugated material rotary pressure valve configured to collect the sized corrugated material, a sized corrugated material storage bin for storing the sized corrugated material, a second conveyor configured to transfer a paper material, a second shredder adjacent to the second conveyor, the second shedder configured to strip cut and cross cut the paper material, a separator located in relation to the second shredder, the separator configured to separate the paper material from fines and dust, wherein the separator includes an after filter configured to separate the paper material from the fines and dust, a paper material rotary pressure valve located in relation to the separator, the paper material rotary pressure valve configured to collect the paper material, a paper material storage bin for storing the paper material, a blending system located in relation to the sized corrugated material storage bin and the paper material storage bin, the blending system including a sized corrugated material variable speed drive auger and a paper material variable speed drive auger, wherein the sized corrugated material variable speed drive auger is located in relation to the sized corrugated material storage bin, the sized corrugated material variable speed drive auger configured to move the sized corrugated material into a mixing bin, wherein the paper material variable speed drive auger is located in relation to the paper material storage bin, the paper material variable speed drive auger configured to move the paper material into the mixing bin, and wherein the blending system proportions the sized corrugated material in relation to the paper material.

### BRIEF DESCRIPTION OF THE DRAWINGS

The above-mentioned and other features of this invention, and the manner of attaining them, will become more apparent and the invention itself will be better understood by reference to the following description of embodiments of the invention taken in conjunction with the accompanying drawings, wherein:

In FIG. 1, an animal bedding material made from an aggregate of paper and corrugated material is shown.

FIG. 2 is a perspective view of the animal bedding material of FIG. 1.

FIG. 3 is a perspective view of the animal bedding material of FIG. 1.

FIG. 4 is a perspective view of an apparatus for producing the animal bedding material of FIG. 1.

### DETAILED DESCRIPTION OF THE EXEMPLARY EMBODIMENTS

The embodiments disclosed below are not intended to be exhaustive or limit the invention to the precise forms disclosed in the following detailed description. Rather, the embodiments are chosen and described so that others skilled in the art may utilize their teachings.

Corresponding reference characters indicate corresponding parts throughout the several views. Although the drawings represent embodiments of the present invention, the drawings are not necessarily to scale and certain features may be exaggerated in order to better illustrate and explain the present invention.

As shown in FIG. 1, a single piece of sized paper 10 and a single piece of sized corrugated material 20, such as cardboard, are illustrated. Animal bedding material of the present disclosure is comprised of aggregate 30 including sized paper 10 and sized corrugated material 20. In one embodiment, aggregate 30 is used in combination with traditional bedding materials. The single pieces of paper 10 and corrugated material 20 are representative of aggregate 30. Paper 10, corrugated material 20, and aggregate 30 are described below in greater detail.

Paper 10 may come from recycled paper, newspaper, shreds of unused paper, and the like. Paper 10 is not expressly limited by its thickness and is generally contemplated to be conventional paper thickness, in an exemplary embodiment, newspaper thickness. Paper 10 may be plain, colored, lined, or the like. In an exemplary embodiment, the source of paper 10 is unused newspaper, also known as over-issue news. In another exemplary embodiment, paper 10 has not been altered, such as wetted, crumpled, or marred. Paper 10 may or may not include text or ink. In yet another exemplary embodiment, paper 10 does not include antibiotic agents. In other embodiments of the present disclosure, antibiotic agents are added by physical, static, or chemical bonding.

Furthermore, sized corrugated material 20 may come from sources such as directly manufactured cardboard, or recycled waste product, including used boxes, posters, and scrap booking material, if the materials meet quality control characteristics.

In an exemplary embodiment, aggregate 30 does not include resin or any fragrant materials such as oils, anti-bacterial compounds, indicator dyes, or other indicator coloring agent medicated compounds and the like.

In yet another embodiment of the present disclosure, pet odor deterrent agents are added, such as Metazine, under non-toxic conditions. In yet another exemplary embodiment, a scent is added to aggregate 30. The scent may be a pine scent or lemon scent. The pine scent may come from oils such as pine oil or pine scented oil and may be attached or impregnated in paper 10, corrugated material 20, and/or aggregate 30. The lemon scent may come from similar sources as the pine scent.

In yet another exemplary embodiment, paper 10 has been modified in size and/or shape by a sizing process. Paper 10 may be sized to length 12. Length 12 may include a range of about 0.25 inches (¼″; 6.35 mm) to about 0.75 inches (¾″; 19.05 mm). In an exemplary embodiment, length 12 is about 0.5625 inches ( 9/16″, 12.7 mm). Paper may be sized to width 14. Width 14 may include a range of about 0.03 inches (0.762 mm) to about 0.09 inches (2.286 mm). In an exemplary embodiment, width 14 is about 0.0625 inches ( 1/16″, 1.5875 mm). Various combinations of size and shape are contemplated within the general teachings of the present disclosure.

Similarly, corrugated material 20 may be sized to length 22. Length 22 may include a range of about 0.25 inches (¼″, 12.7 mm) to about 1.5 inches (3/2″, 38.1 mm). It is recognized that length 22 could be longer based on the type of animal or owner preference. In yet another embodiment length 22 may be based upon the efficiency of the cutting mechanism. In yet another embodiment, length 22 may be based upon not cutting the end of the source of corrugated material 20 or allowing the end of the source of corrugated material 20 to exceed length 22. In an exemplary embodiment, at least some of corrugated material 20 has length 22 which is about 0.75 inches (¾″, 19.05 mm). Furthermore, sized corrugated material 20 may have width 24. Width 24 may include a range of about 0.125 inches (⅛″, 3.175 mm) to about 0.75 inches (¾″, 19.05 mm). In an exemplary embodiment, corrugated material 20 has width 24 which is about 0.4 inches ( 4/10″, 10.16 mm). The thickness of the corrugated material may be sized. There are many variations in size and shape of corrugated material 20 including variations in size and shape of the thickness of corrugated material 20.

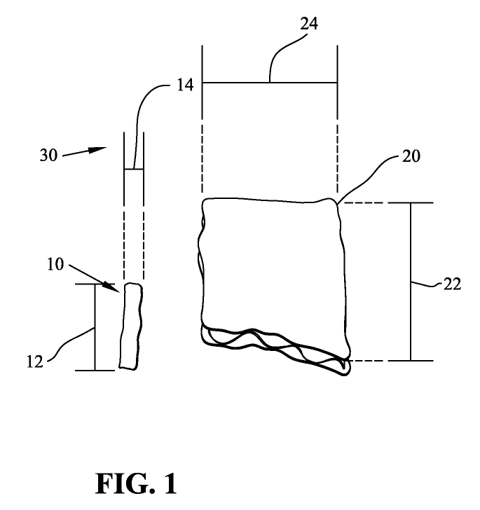

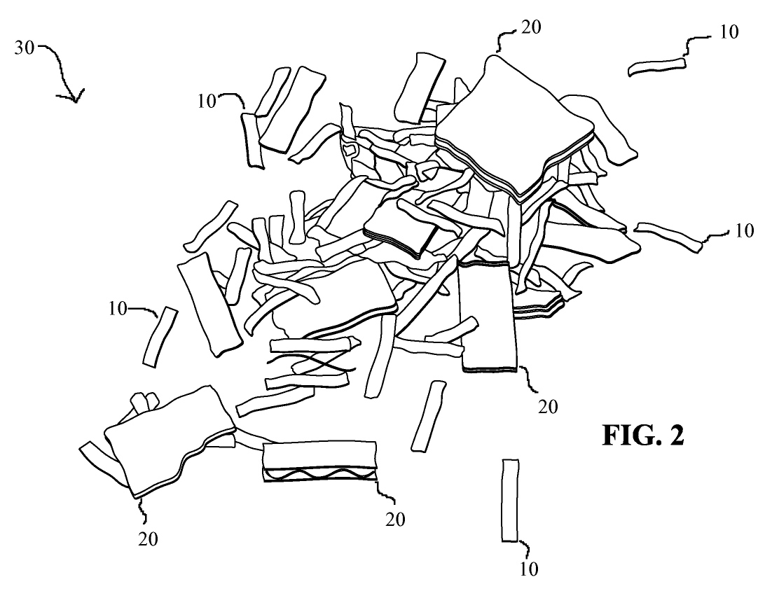

As shown in FIG. 2, aggregate 30 of sized paper 10 and sized corrugated material 20 is illustrated. Aggregate 30 is not limited in the ratio of sized paper 10 and sized corrugated material 20. In an exemplary embodiment, aggregate 30 may have a ratio within the range of about 5-10 pounds of sized paper 10 to about 30-34 pounds of sized corrugated material 20. In another exemplary embodiment, aggregate 30 may have a ratio of about 6:34 pounds of sized paper 10 to sized corrugated material 20. Furthermore, ratios may be determined by weight, volume, concentration, or any permutations between the units such as weight/volume or concentration/weight ratios.

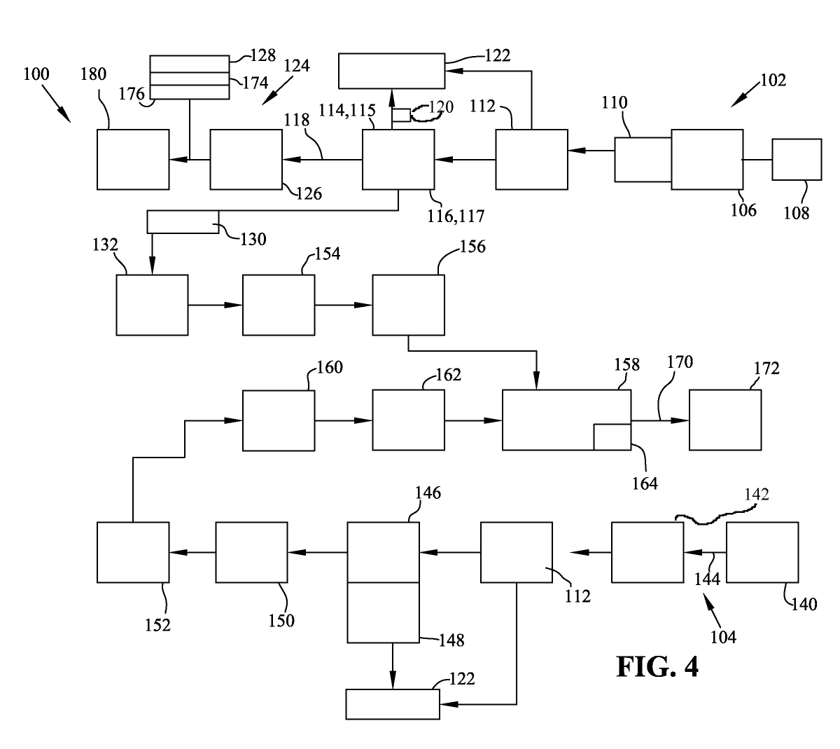

Animal bedding material is comprised of aggregate 30 which includes sized paper 10 and sized corrugated material 20 which are sized and filtered to impede dust generation. Dust generation may lead to respiratory challenges for an animal residing in the animal bedding material. Paper 10 may be sized to minimize creation of dust 122 and fine particles 122 (FIG. 4), also described throughout as fines 122. Fines 122 may become airborne and could effect the animal's respiratory system. Furthermore, paper 10 and corrugated material 20 are sized to provide for efficient collection by methods such as raking. More specifically, paper 10 may act as a dry coating over animal waste. Paper 10 may also lead to less spreading of animal waste which aids in efficient collection.

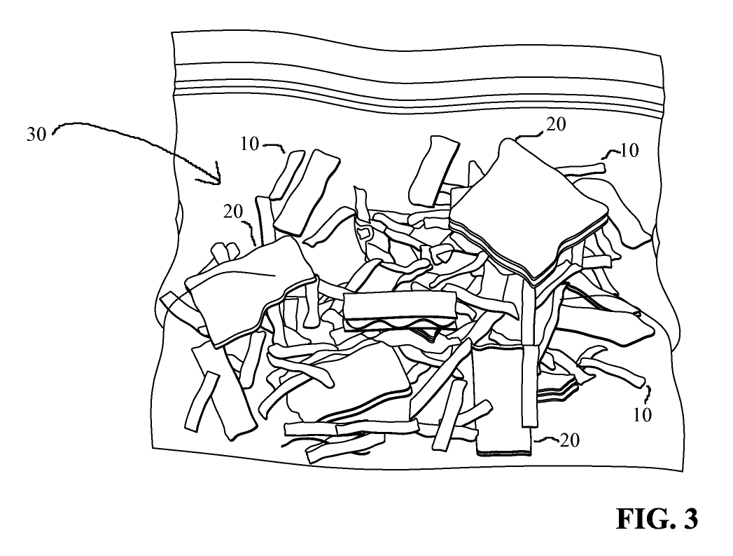

As shown in FIG. 3, aggregate 30 may be bagged or packaged or contained for sale. Furthermore, aggregate 30 may be volume reduced by such methods as vacuum sealing. Optionally, any such baggage, package, or container may also include instructions on how to use aggregate 30. Such instructions may also include how to reduce respiratory infection in animals, how to reduce animal odor, how to efficiently collect the animal bedding material and/or dispose of the same material.

Once unpackaged aggregate 30 can be spread out over an animal bedding area. Aggregate 30 may be aerated by such methods as tilling or turning over. After aggregate 30 has performed as animal bedding material, aggregate 30 including any animal waste matter may be collected or otherwise manipulated for disposal.

As shown in FIG. 4, apparatus 100 is illustrated. Apparatus 100 includes corrugated material pathway 102 and paper material pathway 104. Corrugated material pathway 102 includes corrugated conveyor 106 for transporting corrugated material 108. Corrugated conveyor 106 may be one unit or several units throughout apparatus 100. Apparatus 100 may utilize conveyor 106 to move corrugated material 108 through several parts of apparatus 100. Conveyor 106 may also locate corrugated material 108 in preparation for later processes in apparatus 100.

Corrugated material pathway 102 also includes shredder 110. Shredder 110 is configured to cut, size, and/or shape corrugated material 108 to sized corrugated material 20. In one embodiment, shedder 110 is a cross cut shredder. In this embodiment, shredder 110 is configured to strip cut and then cross cut corrugated material 108 to produce sized corrugated material 20. An exemplary embodiment of shredder 110 is Bedmaster shredder, available at Compac Systems LLC of Holland, Mich.

Corrugated material pathway 102 also includes pneumatic pickup hood 112 for collection of dust 122 and fines 122. Pneumatic pickup hood 112 may include one or more corrugated conveyors 106.

Corrugated material pathway 102 also includes at least one material classifier 114. Classifier 114 includes two sections. Classifier 114 includes separator 116 configured to separate sized corrugated material 20 from large corrugated material 118.

In one embodiment, separator 116 includes walking screen conveyor 117 where sized corrugated material 20 is twice separated from large corrugated material 118. Walking screen conveyor 117 includes a conveyor belt. The conveyor belt defines holes sized to allow sized corrugated material 20 to pass through the conveyor belt. The holes are also sized to restrict large corrugated material 118. The conveyor belt may be vibrated. The conveyor belt may be configured to twice pass through separator 116 and corrugated material pathway 102. Sized corrugated material 20 may continue on corrugated material pathway 102. Large corrugated material 118 may be transported by the conveyor belt to large corrugated material pathway 124.

Classifier 114 also includes filter 120 which filters out dust 122 and fines 122. Several separation processes are envisioned including, for example, sieving, filtration, sedimentation, gravity separation, and winnowing.

Corrugated material pathway 102 may also include optional classifier 115. Classifier 115 is similar to classifier 114. Classifier 115 may be placed in apparatus 100 to increase the efficiency of corrugated material pathway 102. Classifier 115 may minimize the percentage of dust 122 and fines 122 within sized corrugated material 20. Classifier 115 may also minimize the percentage of large corrugated material 118 within sized corrugated material 20.

Corrugated material pathway 102 diverges to large corrugated material pathway 124 for large corrugated material 118. In large corrugated material pathway 124, large corrugated material 118 passes through rotary pressure valve 126 and is transported to large corrugated storage bin 128. Large corrugated material 118 may be stored for future processing as described below.

Similar to large corrugated material 118, sized corrugated material 20 is transported by rotary pressure valve 130 into sized storage bin 132. Sized corrugated material 20 may be stored in sized storage bin 132 for current or future processing as described below.

Paper material pathway 104 is configured to process paper material 140 into sized paper material 10. Paper material pathway 104 includes shredder 142. Shredder 140 is configured to cut, size, and/or shape paper material 140 into sized paper material 10. In one embodiment, shedder 110 is a cross cut shredder. In this embodiment, shredder 110 is configured to strip cut and then cross cut corrugated material 108 to produce sized corrugated material 20. An exemplary embodiment of shredder 110 is Bedmaster shredder, available at Compac Systems LLC of Holland, Mich.

Paper material pathway 104 optionally includes paper conveyor 144 for transporting paper material 140 to shredder 142. Similar to corrugated material pathway 102, paper conveyor 144 may be one unit or several units throughout apparatus 100. Apparatus 100 may utilize paper conveyor 144 to move paper material 140 through several parts of apparatus 100. Conveyor 144 may also locate paper material 140 in preparation for later processes in apparatus 100.

In one embodiment, paper material pathway 104 does not include conveyor 144 prior to paper shredder 142. In this embodiment, paper material 140 is manually fed into paper shredder 142.

Paper material pathway 104 also includes pneumatic pickup hood 112 for collection of dust 122 and fines 122. Pneumatic pickup hood 112 may include one or more corrugated conveyors 106.

Paper material pathway 104 also includes separator 146. Separator 146 includes after filter 148 which separates sized paper material 10 from dust 122 and fines 122. Similar to large corrugated material 118 and sized corrugated material 20, sized paper material 10 is transported by rotary pressure valve 150 into sized paper storage bin 152. Sized paper material 10 can be stored in sized storage bin 152 for current or future processing as described below.

Corrugated material pathway 102 and paper material pathway 104 are, as described so far, configured to process corrugated material 108 and paper material 140 into sized corrugated material 20 and sized paper material 10, respectively. Corrugated material pathway 102 and paper material pathway 104 are also configured to remove dust 122 and fines 122 from sized corrugated material 20 and sized paper material 10, respectively.

At this step of the method and apparatus for processing corrugated material 108 into sized corrugated material 20, sized corrugated material 20 may be stored in sized corrugated storage bin 132. Similarly, at this step of the method and apparatus for processing paper material 140 into sized paper material 10, sized paper material 10 may be stored in sized paper storage bin 152. The previously mentioned method of processing can be performed independent of the later described steps of mixing, baling, and bagging.

Corrugated material pathway 102 includes corrugated auger 154 which is configured to run at a plurality of speeds. In one embodiment, corrugated auger 154 is a variable speed drive auger. Corrugated auger 154 may move sized corrugated material 20 from sized corrugated storage bin 132. Corrugated auger 154 may optionally move sized corrugated material 20 from the bottom of sized corrugated storage bin 132. Corrugated material pathway 102 also includes hopper 156, also known as chute 156, which transfers sized corrugated material 20 into mixing bin 158.

Similarly, paper material pathway 104 includes paper auger 160 which is also configured to run at a plurality of speeds. In another embodiment, paper auger 160 is a variable speed drive auger. Paper auger 160 may move sized paper material 10 from sized paper storage bin 152. Paper auger 160 may optionally move sized paper material 10 from the bottom of sized paper storage bin 152. Paper material pathway 104 also includes hopper 162, also known as chute 162, which transfers sized paper material 10 into mixing bin 158.

Mixing bin 158 includes rotating paddles 164 for mixing sized corrugated material 20 and sized paper material 10 into aggregate 30. Chemical components may be added to mixing bin 158 for blending with aggregate 30 by a chemical adding device such as a spraying device, a drip device, a misting device, etc. It is envisioned that chemical components may be added at several other portions of corrugated material pathway 102 and paper material pathway 104. After aggregate 30 is blended and finished, aggregate 30 is configured to be measured 170, baled, bagged, and shipped 172.

Similarly, large corrugated material 118 includes large corrugated auger 174 which is also configured to run at a plurality of speeds. In one embodiment, large corrugated auger 174 is a variable speed drive auger. Large corrugated auger 174 may move large corrugated material 118 from large corrugated storage bin 128. Large corrugated auger 174 may optionally move large corrugated material 118 from the bottom of large corrugated storage bin 128. Large corrugated material pathway 124 also includes hopper 176, also known as chute 176, which transfers large corrugated material 118 into mixing bin 158.

Large corrugated material 118 may be used with sized paper material 10 to produce large aggregate 180. Some animals may prefer large aggregate 180.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. An animal bedding material for use alone or in combination with other bedding materials, the bedding material comprising
an aggregate of paper and corrugated material,
wherein the paper material is sized to a length in a range of about 0.25 inch to about 0.75 inch and a width in a range of about 0.03 inch to about 0.09 inch,
wherein the corrugated material is sized to a length in a range of about 0.25 inch to about 1.5 inches and a width in a range of about 0.125 inch to about 0.75 inch, and
wherein the paper material and corrugated material is sized to minimize dust and fine particle generation and to provide for efficient absorption of animal waste.

2. The bedding material of claim 1 further comprising a scent added to the aggregate.

3. The bedding material of claim 2 wherein the scent is liquid impregnated.

4. The bedding material of claim 2 wherein the scent is physically mixed.

5. The bedding material of claim 2 wherein the scent is of pine oil and antibiotic.

6. The bedding material of claim 1 wherein the paper material is newspaper shaving.

7. The bedding material of claim 1 wherein the paper material and the corrugated material excludes resin.

8. The bedding material of claim 1 wherein the paper material is sized to a length within the range of about 8/16 inch to about 10/16 inch and a width within the range of about 1/32 inch to about 3/32 inch.

9. The bedding material of claim 1 wherein the corrugated material is sized to a length within the range of about ½ inch to about 1 inch and a width within the range of about 3/10 inch to about 5/10 inch.

10. The bedding material of claim 1 wherein the aggregate includes the paper material within the range of about 5 to about 10 pounds.

11. The bedding material of claim 1 wherein the aggregate includes the sized corrugated material within the range of about 30 to about 34 pounds.

12. The bedding material of claim 1 wherein the aggregate has a ratio of about 6 pounds of the paper material to about 34 pounds of the sized corrugated material.

13. A method of generating animal bedding material comprising the steps of:
sizing paper,
sizing corrugated material, and
aggregating the paper and the corrugated material.

14. The method of claim 13 further comprising the step of adding a scenting agent to the aggregate.

15. The method of claim 14 further comprising the step of adding a scenting agent to the aggregate having a pine scent.

16. The method of claim 13 further comprising the step of packaging the aggregate.

17. The method of claim 13 wherein sizing paper includes the step of forming paper having a length in a range of about 0.25 inch to about 0.75 inch and a width in a range of about 0.03 inch to about 0.09 inch.

18. The method of claim 13 wherein sizing paper includes the step of forming paper having a length within the range of about 8/16 inch to about 10/16 inch and a width within the range of about 1/32 inch to about 3/32 inch.

19. The method of claim 13 wherein sizing corrugated material includes the step of forming corrugated material having a length in a range of about 0.25 inch to about 1.5 inches and a width in a range of about 0.125 inch to about 0.75 inch.

20. The method of claim 13 wherein sizing corrugated material includes the step of forming corrugated material including at least some corrugated material having a length within the range of about ½ inch to about 1 inch and a width within the range of about 3/10 inch to about 5/10 inch.

21. An apparatus for providing an animal bedding material, comprising:
a first shredding station adapted to create animal bedding material including sized corrugated material;
a second shredding station adapted to create animal bedding material from paper; and
a blending system located in relation to said first shredding station and said second shredding station, the blending system including a sized corrugated material variable speed drive auger, a paper material variable speed drive auger, and a mixing bin,
wherein the sized corrugated material variable speed drive auger is located in relation to said first shredding station, the sized corrugated material variable speed drive auger configured to move the sized corrugated material into said mixing bin,
wherein the paper material variable speed drive auger is located in relation to said second shredding station, the paper material variable speed drive auger configured to move the paper material into said mixing bin, and
wherein the blending system proportions the sized corrugated material in relation to the paper material.

22. The apparatus of claim 21 wherein said first shedding station includes:
a first conveyor configured to transfer a corrugated material,
a first shredder adjacent to the first conveyor, the first shedder configured to strip cut and cross cut the corrugated material,
a pneumatic pick up hood adjacent to the first shredder, the pneumatic pick up hood configured to convey the corrugated material,
at least one classifier located in relation to the pneumatic pick up hood, the classifier configured to separate the corrugated material based on the size of the corrugated material so that sized corrugated material is separated from large corrugated material,
a sized corrugated material rotary pressure valve located in relation to the at least one classifier, the sized corrugated material rotary pressure valve configured to collect the sized corrugated material, and
a sized corrugated material storage bin for storing the sized corrugated material.

23. The apparatus of claim 21, wherein said first shredding station includes a cross cut shredder.

24. The apparatus of claim 21 wherein said second shedding station includes:
a second conveyor configured to transfer a paper material,
a second shredder adjacent to the second conveyor, the second shedder configured to strip cut and cross cut the paper material,
a separator located in relation to the second shredder, the separator configured to separate the paper material from fines and dust,
a paper material rotary pressure valve located in relation to the separator, the paper material rotary pressure valve configured to collect the paper material, and
a paper material storage bin for storing the paper material.

25. The apparatus of claim 24, wherein the separator includes an after filter configured to separate the paper material from the fines and dust.

26. The apparatus of claim 21 wherein the blending system includes a chemical component addition system, the chemical component addition system configured to add a chemical component to said mixing bin, the chemical component configured to mix with an aggregate of sized corrugated material and paper material.

27. The apparatus of claim 21, wherein said mixing bin includes rotating paddles.

28. The apparatus of claim 21 further comprising a chemical component adding device for adding a chemical to said mixing bin.

29. The apparatus of claim 21 further comprising a second classifier located in relation to the at least one classifier, the second classifier configured to size and separate the corrugated material,
wherein the second classifier configured to separate the corrugated material based on the size of the corrugated material so that sized corrugated material is separated from large corrugated material, the second classifier configured to twice separate the sized corrugated material from the large corrugated material,
wherein the second classifier includes a second filter configured to separate the sized corrugated material from fines and dust.

30. The apparatus of claim 21, wherein the at least one classifier includes a filter configured to separate the sized corrugated material from fines and dust.

31. The apparatus of claim 21 further comprising a large corrugated material rotary pressure valve located in relation to the at least one classifier, the large corrugated material rotary pressure valve configured to collect the large corrugated material.

32. The apparatus of claim 31 further comprising a large corrugated material storage bin for storing the large corrugated material.

33. A method of using animal bedding material comprising the steps of:
providing an aggregate of paper and corrugated material,
wherein the paper material is sized to a length in a range of about 0.25 inch to about 0.75 inch and a width in a range of about 0.03 inch to about 0.09 inch,
wherein the corrugated material is sized to a length in a range of about 0.25 inch to about 1.5 inches and a width in a range of about 0.125 inch to about 0.75 inch, and
deploying the aggregate for use as the animal bedding material.

34. The method of claim 33 further comprising the step of:
mixing the aggregate with other bedding materials.

35. The method of claim 33 wherein the aggregate is deployed around or adjacent to an animal.

36. The method of claim 33 further comprising the step of:
removing the aggregate and animal waste product.
